(19) United States
(12) Patent Application Publication
(10) Pub. No.: US 2026/0076617 A1
(43) Pub. Date: Mar. 19, 2026

(54) SYSTEMS AND METHODS FOR DETERMINING A RISK FACTOR FOR A CONDITION

(71) Applicant: ResMed Digital Health Inc., San Diego, CA (US)

(72) Inventors: Yang Yan, Singapore (SG); Michael Christopher Hogg, Sydney (AU); Gregory Robert Peake, Sydney (AU); Albert Jack Greenwood Woffenden, Sydney (AU); Priyanshu Gupta, Hornsby, NSW (AU); Stewart Joseph Wagner, Sydney (AU); Garth Alan Berriman, Sydney (AU); Jamie Graeme Wehbeh, Bella Vista, NSW (AU); Zhou Qi Lee, Singapore (SG)

(21) Appl. No.: 19/104,141

(22) PCT Filed: Aug. 10, 2023
(86) PCT No.: PCT/US2023/029967

§ 371 Date: Feb. 14, 2025

Related U.S. Application Data
(60) Provisional application No. 63/398,831, filed on Aug. 17, 2022.

(51) Int. Cl. A61B 5/00; A61B 7/00; G06T 7/00; G06V 10/70; G06V 40/10; G16H 50/30

(52) U.S. Cl. CPC A61B 5/4818; A61B 5/0064; A61B 5/0088; A61B 5/4803; A61B 5/7275; A61B 7/003; G06T 7/0016; G16H 50/30; G06T 2207/30004; G06T 2207/30196; G06V 10/70; G06V 40/10; G06V 2201/03

(57) ABSTRACT

A method for determining a risk factor for a condition comprises generating first image data of the interior of the mouth of an individual. The first image data is associated with one or more internal physical features of the individual. The method further comprises generating second image data of the exterior of the mouth of an individual. The second image data is associated with one or more external physical features of the individual. The method further comprises determining a risk factor for the individual associated with the condition that is based at least in part on the first image data, the second image data, or both.

### CROSS-REFERENCE TO RELATED APPLICATIONS

This application claims the benefit of, and priority to, U.S. Provisional Patent Application No. 63/398,831 filed on Aug. 17, 2022, which is hereby incorporated by reference herein in its entirety.

### TECHNICAL FIELD

The present disclosure relates generally to systems and methods for determining a risk factor for a condition, and more particularly, to systems and methods for determining a risk factor for a condition based on internal and/or external physical features of an individual.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. The pressurized air is delivered via at least a conduit coupled to a respiratory therapy device of the respiratory therapy system, and a user interface that is worn by the individual. Various different physical features, both internal and external, can impact the development and/or severity of these disorders, as well as the efficacy of treatment via the respiratory therapy system. Thus, it would be advantageous to be able to accurately monitor physical features of the individual and determine the individual's risk in developing any of these disorders. The present disclosure is directed to solving these and other problems.

### SUMMARY

According to some implementations of the present disclosure, a method for determining a risk factor for an individual that is associated with a condition includes generating first image data of an interior of a mouth of the individual, an interior of a throat of the individual, or both. The first image data is associated with one or more internal physical features of the individual. The method further includes determining a risk factor for the individual associated with a condition, based at least in part on the first image data. In some implementations, determining the risk factor can include determining whether the individual currently has the condition, determining a likelihood that the individual will develop the condition, or both. In some implementations, the method further includes generating second image data of a head of the individual, a neck of the individual, or both. The second image data is associated with one or more external physical features of the individual. In some implementations, the risk factor can be based on only the first image data, based on only the second image data, based on both the first image data and the second image data, initially based on the first image data and then updated based on the second image data, initially based on the second image data and then updated based on the first image data, or based on the first image data and/or the second image data as well as additional image data.

According to some implementations of the present disclosure, a system for determining a risk factor for an individual that is associated with a condition includes an electronic interface, a memory, and a control system. The electronic interface is configured to receive and/or generate data associated with the individual. The memory stores machine-readable instructions. The control system includes one or more processors configured to execute the machine-readable instructions to generate first image data of an interior of a mouth of an individual, an interior of a throat of the individual, or both. The first image data is associated with one or more internal physical features of the individual. The one or more processors are further configured to execute the machine-readable instructions to determine a risk factor for the individual based at least in part on the first image data. In some implementations, determining the risk factor can include determining whether the individual currently has the condition, determining a likelihood that the individual will develop the condition, or both. In some implementations, the one or more processors are further configured to execute the machine-readable instructions to generate second image data of a head of the individual, a neck of the individual, or both. The second image data is associated with one or more external physical features of the individual. In some implementations, the risk factor can be based on only the first image data, based on only the second image data, based on both the first image data and the second image data, initially based on the first image data and then updated based on the second image data, initially based on the second image data and then updated based on the first image data, or based on the first image data and/or the second image data as well as additional image data.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A is a perspective view of an individual generating image data associated with one or more internal physical features, according to some implementations of the present disclosure;

FIG. 5B is a perspective view of an individual generating image data associated with one or more external physical features, according to some implementations of the present disclosure;

FIG. 6 is a flow diagram of a method for determining a risk factor, according to some implementations of the present disclosure; and

FIG. 7 is a diagram of five possible Mallampati score classes, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate, and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea. As will be understood, a sleep session as described herein can alternatively be referred to as a therapy session, during which an individual may receive respiratory therapy, or can comprise or consist of a therapy session.

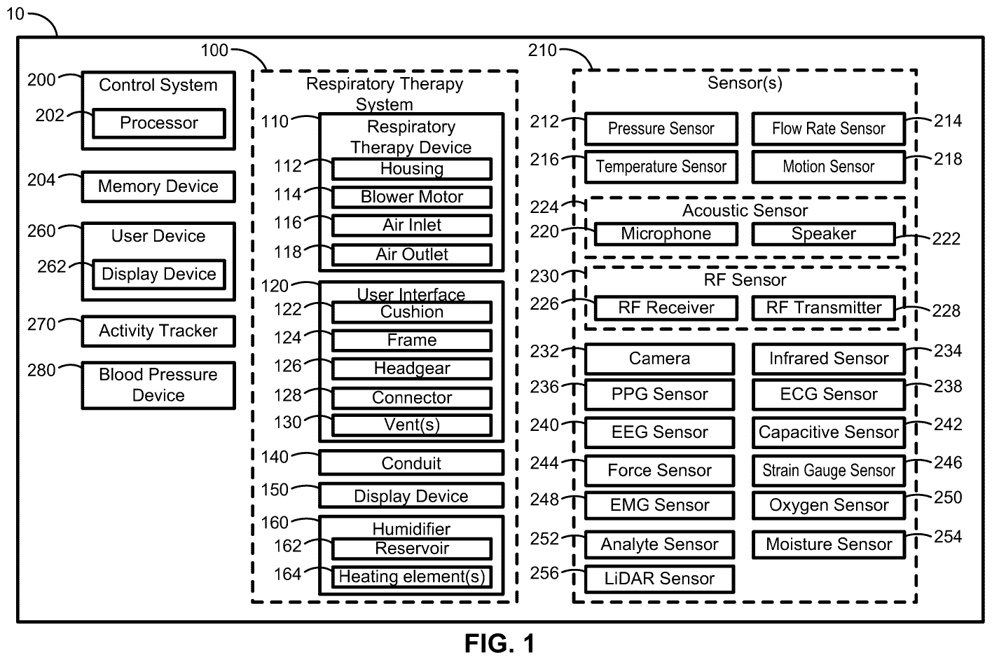

Referring to FIG. 1, a system 10, according to some implementations of the present disclosure, is illustrated. The system 10 can include a respiratory therapy system 100, a control system 200, a memory device 204, and one or more sensors 210. The system 10 may additionally or alternatively include a user device 260, an activity tracker 270, and a blood pressure device 280. The system 10 can be used to analyze data from an individual and determine a risk factor for the individual that is associated with a condition.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

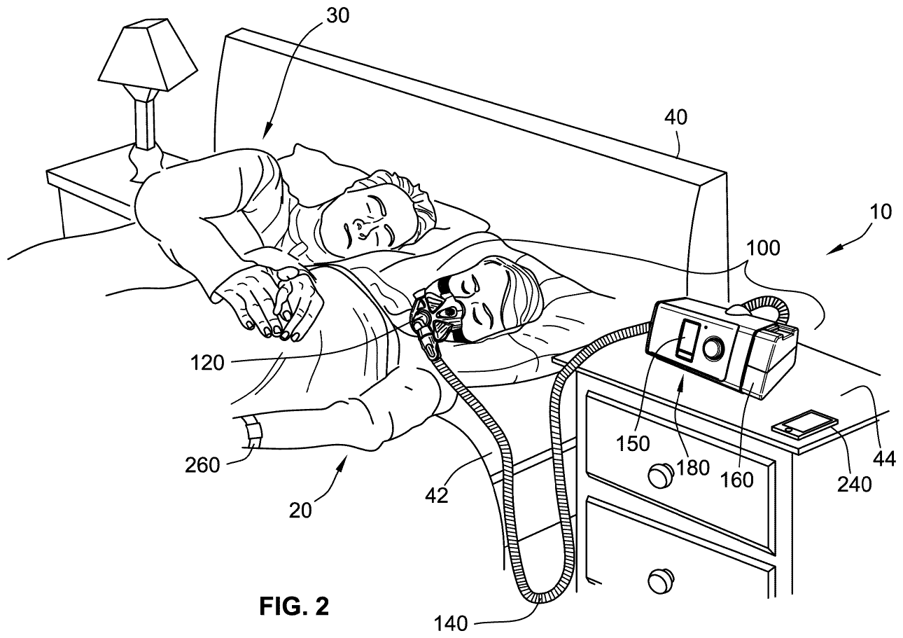

As shown in FIG. 2, the respiratory therapy system 100 can be used to treat a user 20. In this example, the user 20 of the respiratory therapy system 100 and a bed partner 30 are in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

Referring back to FIG. 1, the respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

The respiratory therapy device 110 includes a housing 112, a blower motor 114, an air inlet 116, and an air outlet 118. The blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). The housing 112 can also include a vent to allow air to pass through the housing 112 to the air inlet 116. As described below, the conduit 140 is coupled to the air outlet 118 of the respiratory therapy device 110.

The user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The one or more vents 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full-face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

The conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flows from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid-state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct). Thus, while the control system 200 and the memory device 204 are shown as independent components in the block diagram of FIG. 1, they may be components of some other component of the system 10, such as the user device 260, the respiratory therapy device 110, etc.

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

The one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared (IR) sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a Light Detection and Ranging (LiDAR) sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the IR sensor 234, the PPG sensor 236, the ECG sensor 238, the EEG sensor 240, the capacitive sensor 242, the force sensor 244, the strain gauge sensor 246, the EMG sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleep stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20, a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 can comprise an acoustic sensor (such as the acoustic sensor 224 discussed herein) and/or an RF sensor (such as the RF sensor 230 discussed herein), which can generate motion data as further discussed herein. In such implementations, the motion sensor 218, the acoustic sensor, and/or the RF sensor can be disposed in a portable device, such as the user device 260 or the portable device 550 discussed herein. Further, while FIG. 1 and FIG. 2 show the respiratory therapy device 110 as including its own display device 150, in some implementations the respiratory therapy device 110 may not include its own display device, as is discussed herein. In some implementations, the motion sensor 218 alternatively or additionally generates one or more signals representing bodily movement of the user, from which may be obtained a signal representing a sleep state of the user, for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. The microphone 220 can be coupled to or integrated in a wearable device, such as a smartwatch, smart glasses, earphones or earbuds, or other head-wearable devices. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones.

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260, and/or can be coupled to or integrated in a wearable device, such as a smartwatch, smart glasses, earphones or ear buds, or other head-wearable devices.

The microphone 220 and the speaker 222 can be used as separate devices. In some implementations, the microphone 220 and the speaker 222 can be combined into an acoustic sensor 224 (e.g., a sonar sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30. Based at least in part on the data from the microphone 220 and/or the speaker 222, the control system 200 can determine a location of the user 20 and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g., a radar sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user, to determine air flow of the mouth and/or nose of the user, to determine a time when the user enters the bed, and to determine a time when the user exits the bed. In some implementations, the camera 232 includes a wide-angle lens or a fisheye lens.

The IR sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120, in the associated headgear (e.g., straps, etc.), in a head band or other head-worn sensor device, etc.

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 252 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 252 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The LiDAR sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 214 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, a microphone, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be non-physiological parameters. Other types of physiological and non-physiological parameters can also be determined, either from the data from the one or more sensors 210, or from other types of data.

The user device 260 includes a display device 262. The user device 260 can be, for example, a mobile device such as a smartphone, a tablet computer, a gaming console, a smartwatch, a laptop computer, or the like. In some implementations, the user device 260 is a portable device, such as a smartphone, a tablet computer, a smartwatch, a laptop computer, etc. Alternatively, the user device 260 can be an external sensing system, a television (e.g., a smart television), or another smart home device (e.g., a smart speaker(s) such as Google Home, Amazon Echo, Amazon Alexa, etc.). In some implementations, the user device is a wearable device (e.g., a smartwatch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 10 also includes the activity tracker 270. The activity tracker 270 is generally used to aid in generating physiological data associated with the user. The activity tracker 270 can include one or more of the sensors 210 described herein, such as, for example, the motion sensor 218 (e.g., one or more accelerometers and/or gyroscopes), the PPG sensor 236, and/or the ECG sensor 238. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 10 also includes the blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp, chirp, triangle, sinewave, etc.), other schemes such as PSK, FSK etc., pulsed continuous wave, and/or spread in ultra-wideband ranges (which may include spreading, PRN codes or impulse systems).

While the control system 200 and the memory device 204 are described and shown in FIG. 1 as being a separate and distinct component of the system 10, in some implementations, the control system 200 and/or the memory device 204 are integrated in the user device 260 and/or the respiratory therapy device 110. Thus, the control system 200 and/or the memory device 204 can be disposed within the housing 112 of the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 10 is shown as including all the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

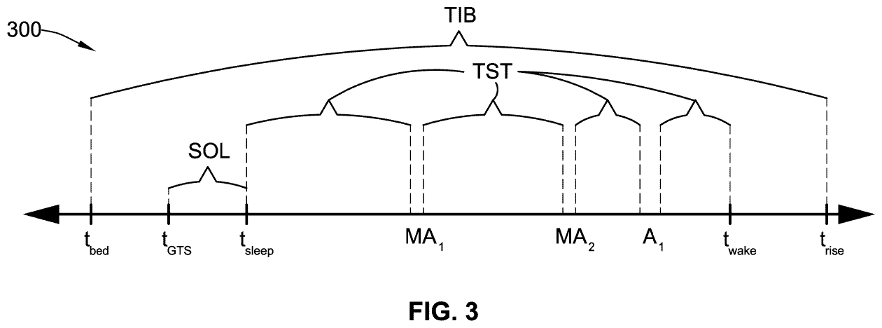

Referring now to FIG. 3, as used herein, a sleep session can be defined multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user has laid or sat down in the bed (or another area or object on which they intend to sleep) and has turned on the respiratory therapy device 110 and donned the user interface 120. The sleep session can thus include time periods (i) when the user is using the respiratory therapy system 100, but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session may also be referred to as a therapy session, or may comprise a therapy session, which can be understood to be the period of time within the sleep session during which the individual is engaged in respiratory therapy (e.g., the use of a respiratory therapy system).

The sleep session is generally defined as ending once the user removes the user interface 120, turns off the respiratory therapy device 110, and gets out of bed. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user, ending when the respiratory therapy device 110 stops supplying the pressurized air to the airway of the user, and including some or all the time points in between, when the user is asleep or awake.

FIG. 3 illustrates an exemplary timeline 300 for a sleep session. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 40 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}), or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, as shown in the timeline 300, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

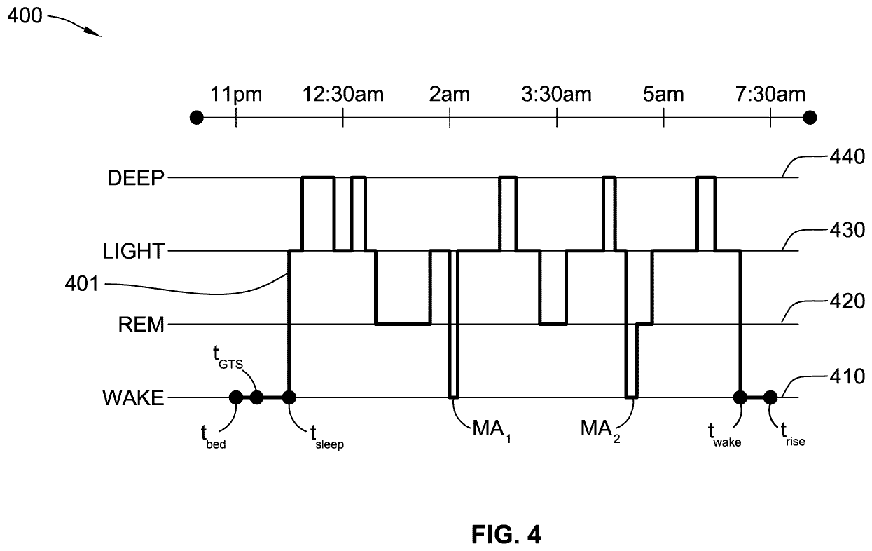

Referring to FIG. 4, an exemplary hypnogram 400 corresponding to the timeline 300 of FIG. 3, according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 210 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 204.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 218, the microphone 220, the camera 232, or any combination thereof. The go-to-sleep time can be determined based at least in part on, for example, data from the motion sensor 218 (e.g., data indicative of no movement by the user), data from the camera 232 (e.g., data indicative of no movement by the user and/or that the user has turned off the lights), data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

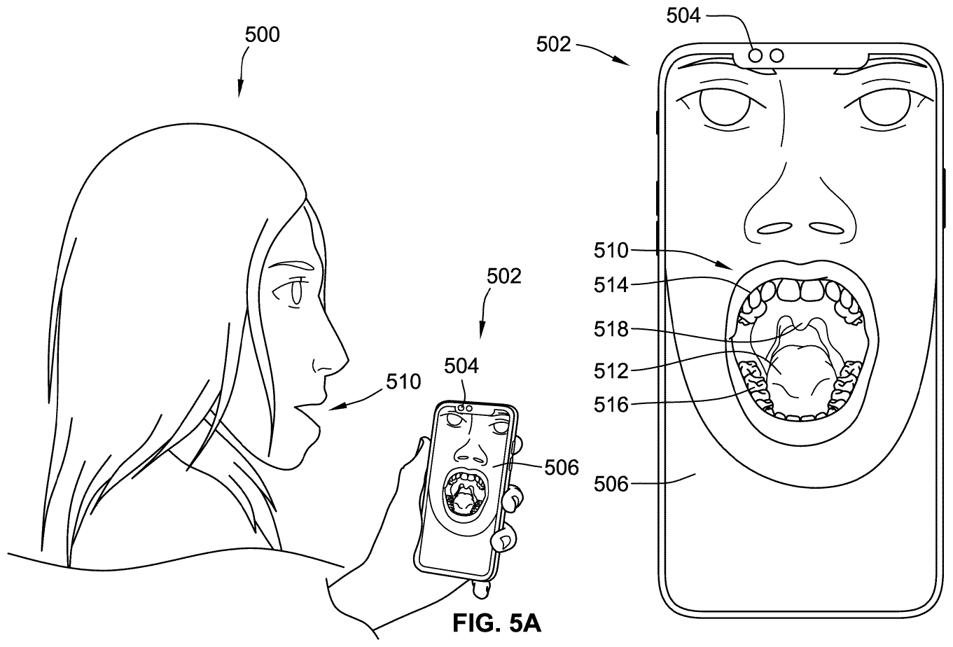

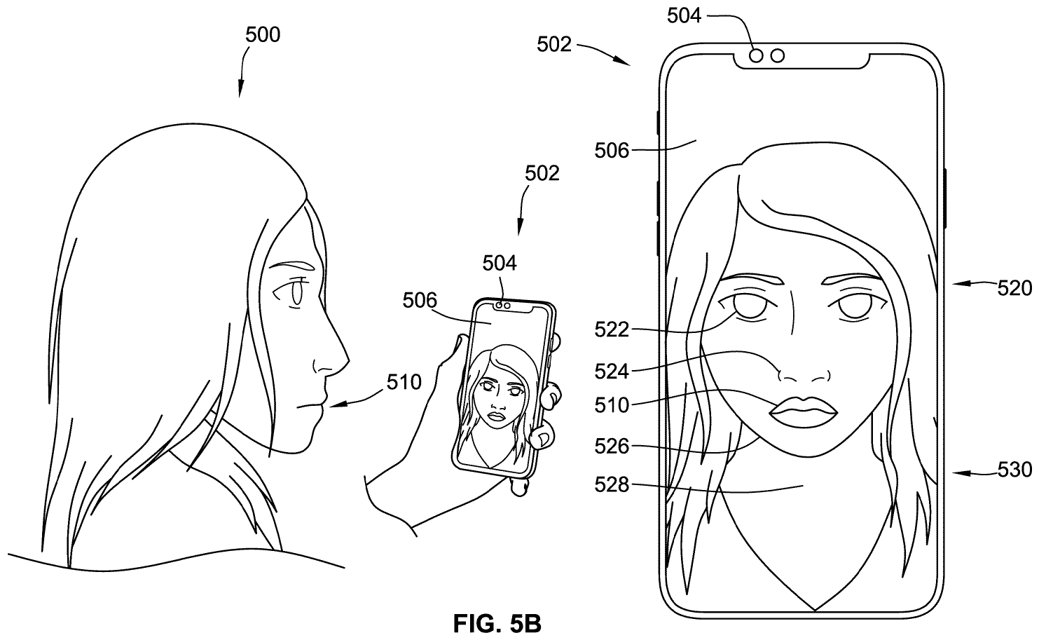

Referring now to FIGS. 5A and 5B, it can be advantageous to be able to analyze various different physical features of an individual to determine the individual's risk factor that is associated with some condition (such as SDB and/or OSA). FIG. 5A shows an individual 500 that is holding a smartphone 502. The smartphone 502 includes an image sensor 504 that is generally aimed in the direction of the individual 500. The smartphone 502 includes a display 506 that can display an image and/or a video of anything in the field of view of the image sensor 504. In FIG. 5A, a mouth 510 of the individual 500 is open, and an image and/or video of the mouth 510 is shown on the display 506 of the smartphone 502. As can be seen, the image/video of the mouth 510 shown on the display 506 includes the individual 500's tongue 512, upper teeth 514, lower teeth 516, and uvula 518. The individual 500 is generally holding the smartphone 502 close enough to their face such that only the mouth 510 is shown in the display 506 of the smartphone 502. In this position, the image sensor 504 can generate image data that is reproducible as one or more images and/or videos of the interior of the mouth 510 of the individual 500. This image data is associated with various different internal physical features of the individual 500 (e.g., the size of the tongue 512, the relative positions of the upper teeth 514 and the lower teeth 516, etc.).

In FIG. 5B, the mouth 510 of the individual 500 is closed, and the smartphone 502 is being held further away from the individual 500's face. As shown on the display 506 of the smartphone 502, the head 520 of the individual 500 is in the field of view of the image sensor 504, but the interior of the mouth 510 is not. Thus, the display 506 of the smartphone 502 shows details of the exterior of the head 520, including the eyes 522, the nose 524, the exterior of the mouth 510, the jaw 526, the throat 528, and the neck 530. In this position, the image sensor 504 can generate image data that is reproducible as one or more images and/or videos of the exterior of the head 520 and/or neck 530 of the individual 500. This image data is associated with various different external physical features of the individual 500 (e.g., the shape of the jaw 526, the circumference of the neck 530, etc.).

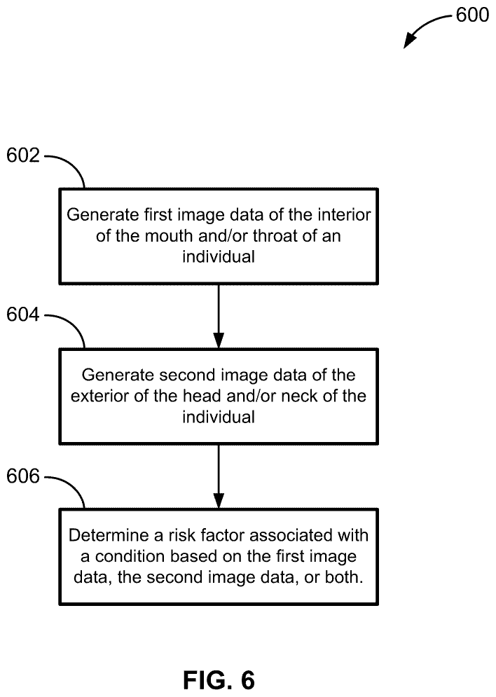

FIG. 6 illustrates a method 600 for determining a risk factor for the individual that is associated with a condition, such as sleep-disordered breathing (SDB) and/or obstructive sleep apnea (OSA). Generally, a control system (such as the control system 200 of the system 10) is configured to carry out the various steps of method 600. A memory device (such as the memory device 204 of the system 10) can be used to store any type of data utilized in the steps of method 600 (or other methods). In some implementations, the risk factor is indicative of the likelihood that the individual will develop the condition at some point in the future. In some implementations, the risk factor is indicative of the likelihood that the individual has already developed the condition. In some implementations, the risk factor is indicative of the severity of condition that the individual has already developed or is at risk of developing.

At step 602 of method 600, first image data of the interior of the individual's mouth and/or throat is generated. The first image data is reproducible as one or more images and/or videos of the interior of the individual's mouth and/or throat, and is indicative of one or more internal physical characteristics of the individual. The first image data can be generated using any suitable device with one or more image sensors, such as a user device (which may be the same as or similar to the user device 260 of the system 10). The user device could be the individual's smartphone, tablet computer, camera, or any other suitable device.

Generally, a variety of different portions of the interior of the individual's mouth and/or throat will in the field of view of the image sensors of the device, such as the tongue, the upper teeth, the lower teeth, the uvula, the gums, the roof of the individual's mouth (also known as the palate, and can include the hard palate and soft palate, the tonsils, the salivary glands, and the mouth cavity (e.g., the open space defined between at least the tongue, the soft palate and/or hard palate, and the inside of the individual's cheeks).

Thus, the first image data can be analyzed to determine a variety of different internal physical features of the individual, such as the size of the individual's tongue (e.g., height, length, etc.), the distance between the individual's tongue and the roof of the individual's mouth, the position of the individual's upper teeth and lower teeth relative to each other and/or other landmarks, the position of the individual's upper jaw and lower jaw relative to each other and/or other landmarks, the width of the individual's upper jaw and lower jaw, the size of the individual's uvula, the height and width of the back of the individual's mouth and/or throat past the uvula, and other features. Generally, the internal physical features that can be identified and/or analyzed with the first image data are physical features of the individual that are not externally visible (e.g., are not within the field of view of the image sensors of the device that is being used to generate the first image data) when the individual's mouth is closed.

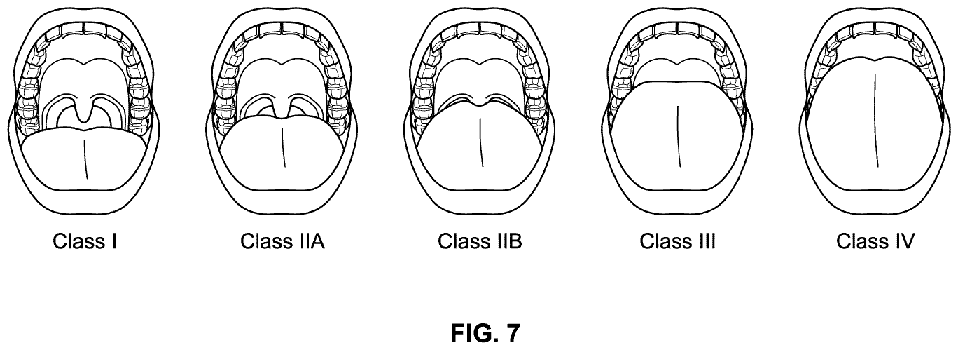

In some implementations, the internal physical features can include a Mallampati score, which is used to classify the amount of open space within the individual's mouth. The Mallampati score is assessed by analyzing the individual's mouth when the individual opens their mouth and extends their tongue. The Mallampati score refers to the amount of anatomical structures that are visible. In some implementations, the individual's Mallampati score is Class I, Class II, Class III, or Class IV. Class I refers to an individual where the soft palate, the hard palate, the uvula, and the tonsils are visible. In some cases, Class I refers to an individual with these features visible, as well as the fauces (e.g., the opening at the back of the mouth into the throat), and faucial pillars (the palatogloassal arches and the palatopharyngeal arches). Class IIA refers to an individual where the soft palate, the hard palate, and most of the uvula is visible. In some cases, Class IIA refers to an individual with these features visible, as well as the fauces. Class IIB refers to an individual where the soft palate, the hard palate, and the base of the uvula are visible. Class IV refers to an individual where only some of the soft palate is visible, along with the hard palate. Class V refers to an individual where only as the hard palate visible. The different classes are shown in FIG. 7, which shows images of an open mouth in each of the five different classes. In some cases, the Mallampati score may only include four classes (I, II, III, and IV). In these implementations, Class II generally refers to an individual where the soft palate and most of the uvula are visible, while Class III generally refers to an individual where the soft palate and only the base of the uvula are visible. Class I and Class IV are generally the same in both of these implementations. In addition to or alternatively to, individual aspects of the Mallampati score may also be included as part of the determined internal physical features. For example, a distinct physical feature could be the size of the uvula, the amount of the uvula that is visible, the size of the soft palate, the amount of the soft palate that is visible, the size of the hard palate, the amount of the hard palate that is visible, the size of the tonsils, the amount of the tonsils that are visible, etc.

The first image data can be analyzed to quantify the internal physical features in a variety of different manners. In some implementations, the absolute value of any identified internal physical features is determined. In other implementations, the value of any identified internal physical feature relative to some baseline is determined. This baseline can be the individual at a prior time, the average of a population of individuals to which the individual belongs, or other baselines.

At step 604 of method 600, second image data of the exterior of the individual's head and/or neck is generated. The second image data is reproducible as one or more images and/or videos of the exterior of the individual's head and/or neck, and is indicative of one or more external physical characteristics of the individual. Similar to the first image data, the second image data can be generated using any suitable device with one or more image sensors, such as a user device (which may be the same as or similar to the user device 260 of the system 10). The user device could be the individual's smartphone, tablet computer, camera, or any other suitable device.

Generally, a variety of different portions of the exterior of the individual's head and/or neck will in the field of view of the image sensors of the device, such as the user's eyes, nose, mouth, chin, jaw, and neck. The second image data can be analyzed to determine a variety of different external physical features of the individual, such as the position and/or size (e.g., width) of the individual's jaw (a smaller jaw size may indicate a weak jaw, which can cause the individual's tongue to fall backward toward the individual's airway when the individual is sleeping on their back), the circumference of the individual's neck, the location and/or amount of body fat in the head and neck area of the individual, the location and/or amount of muscle in the head and neck area of the individual, the size and/or shape of the individual's nose (which may be indicative of nasal congestion or obstruction, which in turn can contribute to SDB), and other features. Physical features related to the individual's jaw can include the alignment of the temporomandibular joint (certain alignments of the temporomandibular joint can impact the tongue's position during sleep, which can then cause the tongue to partially or fully block the individual's airway). Generally, the external physical features that can be identified and/or analyzed with the second image data are physical features of the individual that are externally visible (e.g., are within the field of view of the image sensors of the device that is being used to generate the second image data) when the individual's mouth is closed.

Similar to the internal physical features, the second image data can be analyzed to quantify the external physical features in a variety of different manners. In some implementations, the absolute value of any identified external physical features is determined. In other implementations, the value of any identified external physical feature relative to some baseline is determined. This baseline can be the individual at a prior time, the average of a population of individuals to which the individual belongs, or other baselines.

At step 606 of method 600, the individual's risk factor for a condition is determined. In some implementations, the individual's risk factor is not based on the second image data and the external physical features, and instead is based on the first image data and the internal physical features (and any other information that may be needed). In these implementations, step 604 of method 600 is generally optional, and method 600 may only include generating the first image data. In other implementations, the individual's risk factor is not based on the first image and the internal physical features, and instead is based on the second image data and the external physical features (and any other information that may be needed). In these implementations, step 602 of method 600 is generally optional, and method 600 may only include generating the second image data. In further implementations, the individual's risk factor is based on both the first image data and the second image data (and any other information that may be needed), and thus both steps 602 and 604 will be performed. In these implementations, the initial risk factor can be determined based on the first image data and the second image data, or the initial risk factor can be determined based on either the first image data or the second image data and then updated based on the image data.

In implementations where the risk factor is determined based on just the first image data or just the second image data, the risk factor may be updated based on the other image data. For example, if the determination of the initial risk factor based on only the first image data or only the second image data is not sufficiently accurate, then the initial risk factor can then be updated based on the second image data or the first image data. Generally, the updated risk factor will be more accurate than the initial risk factor.

As noted herein, the individual's risk factor can take different forms. In some implementations, the risk factor is an estimate of whether the individual will develop the condition, which may be expressed as a percentage. In other implementations, the risk factor is an estimate of when the individual will develop the condition, which may be expressed as an absolute time (e.g., on a certain date) or a relative time (e.g., within a certain number of months from the current day). In further implementations, the risk factor is an estimate of whether and when the individual will develop the condition, which may be expressed as both a percentage and a relative time (e.g., there is an X % chance the individual will develop the condition within Y months). In additional implementations, determining the risk factor includes determining that the user has (or likely has) already developed the condition, and estimating the severity of the condition.

In some implementations, determining the risk factor at step 602 can include inputting the first image data and/or the second image data into a trained machine learning model that has been trained to output the risk factor. In some cases, additional information may be input into the trained machine learning model that can be used in conjunction with the first image data and/or the second image data to determine the risk factor. This additional information can include physical characteristics of the individual, such as the individual's height, weight, age, etc. The additional information could additionally or alternatively include information related to the individual's medical history.

In some implementations, method 600 can further include analyzing the physical features of the individual in order to determine what physical features may be causing the individual to be in danger of developing the condition, or causing the individual to have already developed the condition. Thus, method 600 can include determining a contribution degree of any one or more of the physical features (e.g., one or more internal physical features, one or more external physical features, or both). The contribution degree of a respective physical feature represents an estimate of the impact that the respective physical feature has on the risk factor and/or the presence of the condition.

In some implementations, the estimate of the impact is an estimate of how much the respective physical feature has contributed to the development of the condition in the individual (or the likely development of the condition in the individual, if the individual has not yet developed the condition). In some implementations, the estimate of the impact is an estimate of how much the respective physical feature has contributed to the severity of the condition in the individual (or how much the respective physical feature will likely contribute to the severity of the condition, if the individual has not yet developed the condition). Thus, a physical feature with a relatively larger contribution degree is estimated to contributed more to the development and/or severity of the condition than a physical feature with a relatively smaller contribution degree.

In some implementations, the contribution degree is expressed as a percentage (e.g., a given physical feature could be 40% responsible for the development of the condition in the individual). In other implementations, the contribution degree is expressed in relative terms. In these implementations, physical features of the individual (internal, external, or both) can be ranked according to their contribution to the development and/or severity of the condition.

The contribution degree could be determined in a variety of different manners. In some implementations, the contribution degree of a respective physical feature is based at least in part on the deviation of the value of that respective physical feature from some baseline value. The more that the current value of the respective physical feature deviates from the baseline value of the physical feature, the larger the contribution degree of the respective physical feature will be. The baseline value of the respective physical feature could be a previously determined value of the respective physical feature for the individual, an average value for the respective physical feature across a plurality of other individuals within a population of similar individuals, or other values. The contribution degree of the respective physical feature could also be determined by comparing the value of the respective physical feature to some measure of the condition in the individual, such as an AHI measurement. The contribution degree of the respective physical feature could also be determined via machine learning techniques, such as trained models.

The contribution degree of the physical features can be used for a variety of different purposes, including providing the individual with more information about the condition, generating or updating a treatment plan for the condition, and other purposes. In some implementations, physical features having a contribution degree of a threshold contribution degree are identified. For example, analysis of the first image data and/or the second image data may indicate that numerous physical features contributed to the development and/or severity of the condition in the individual (or are contributing to the risk of the individual developing the condition). However, it may not be of much use to the individual to know every single physical feature that is contributing to the development and/or severity of the condition. Thus, physical features with a contribution degree above the threshold (e.g., the main physical features) can be identified, and can be communicated to the individual and/or to a third party, used to development a treatment plan, etc. In some implementations, the identified physical features with a contribution degree above the threshold can be further sorted into whether they are associated with the tongue and/or jaw of the individual (e.g., within the individual's mouth), or whether they are associated with the individual's airway (e.g., from the back of the individual's throat down into the trachea and lungs).

In some implementations, method 600 includes determining whether various different physical features are easily modifiable by the individual. These implementations include making this determination for one or more physical features (internal and/or external), for one or more internal physical features, for one or more external physical features, for one or more physical features having a contribution degree above the threshold (internal and/or external), for one or more internal physical features having a contribution degree above the threshold, for one or more external physical features having a contribution degree above the threshold, or for any other set or sub-set of physical features.

In some of these implementations, a physical feature is considered to be modifiable if it can be modified in response to a change in a physical activity regimen of the individual, a change in a diet of the individual, a change in a medication regiment of the individual, or any combination thereof. The change in any of these regimens can include modifying an existing regimen and beginning a new regimen. The physical activity regimen can include traditional exercise (e.g., strength training, cardiovascular exercise, etc.), but can also include other types of physical activities, such as performing breathing exercises and playing wind instruments (which can aid in the strengthening of neck muscles and/or causing hypertrophy of the neck muscles). In some implementations, modifiable physical features can include the circumference of the individual's neck, the amount and/or location of body fat in the individual's head and/or neck area, the amount and/or location of muscle in the individual's head and/or neck area (e.g., muscles in the tongue and/or upper airway, the strengthening of which can aid in reducing the severity of the condition), and other features.

A physical feature that is considered to be non-modifiable will generally not be able to be modified in response to modifying a physical activity regimen, a diet regimen, or a medication regimen. However, these physical features may be able to be modified in response to more severe interventions, such as surgery. The non-modifiable physical features can include the position and/or size of the individual's jaw, the size of the individual's tongue, the distance between the individual's tongue and the roof of the individual's mouth, the relative positions between the individual's upper teeth and lower teeth, and other features.

The various different physical features can be used to design and recommend a treatment plan for the individual. The treatment can be based on which features are determined to be modifiable and which features are determined to be not modifiable. For example, if it is determined that the main physical features contributing to the development and/or severity of the condition in the individual (e.g., physical features with relatively large contribution degrees) are modifiable (e.g., a large neck circumference due to body fat, weak neck muscles, etc.), the treatment plan may include less severe interventions such as exercise and diet changes. If the treatment plan includes the use of a respiratory therapy system during sleep session, the treatment plan may recommend using the respiratory therapy system with a less aggressive (e.g., lower) therapy pressure. However, if it is determined that the main physical features contributing to the development and/or severity of the condition in the individual are not modifiable (e.g., jaw position contributing to the development of the condition), the treatment plan may include more invasive aspects, such as a more aggressive use of a respiratory therapy system, surgical interventions, etc.

In some implementations, recommending the treatment plan to the individual can include transmitting an explanation of what physical features are contributing to the development and/or severity of the condition in the individual, and how the treatment plan could be modified in the future based on those physical features. For example, in certain cases, individuals may be more receptive to beginning and/or following the treatment plan if they are told that the cause or causes of their condition can be modified more easily by following the treatment plan. In other cases, individuals may be more receptive to beginning and/or following the treatment plan if they know that in the future, the aggressiveness of the treatment may be reduced because the cause or causes of their condition can be modified via physical activity/diet/medication. In even further cases, letting individuals know that the cause or causes of their condition are less able to be controlled by them (e.g., the development of their condition is less their fault) can result in the individuals being more receptive to beginning and/or following the treatment plan.

In some implementations, the physical features of the individual can be monitored over time to determine whether any physical features (e.g., modifiable physical features) have changed, and whether the current treatment plan can or should be modified. For example, the first image data and/or the second image data can be generated and analyzed at a first time to determine the individual's risk factor, and to determine an initial treatment plan. After a certain amount of time of the individual following the treatment plan, additional first image data and/or additional second image data can be generated and analyzed to determine if any of the physical features have changed. If any physical features have changed, the risk factor can be updated based on the changes in the physical features. If the risk factor has been reduced (e.g., the individual now has a smaller chance of developing the condition, the individual's condition is now less severe, etc.), the treatment plan can be updated. The treatment plan could be updated to include a less aggressive use of the respiratory therapy system, less medication, less recommended physical activity, a less restrictive diet, etc.

In some examples, the changes in the physical features may indicate that the individual has lost weight. For example, analysis of the updated image data may indicate that the individual's neck circumference has decreased, and/or that the individual has less body fat in their neck are. Based on these changed physical features, the treatment plan for the condition can be updated. For example, the treatment plan could be updated to include a less aggressive use of the respiratory therapy system (e.g., using the respiratory therapy system with a lower therapy pressure, not using the respiratory therapy system at all, etc.). In another example, the individual's weight loss can result in the individual no longer breathing primarily through their mouth during the sleep session. Thus, the updated treatment plan can include the use of a nasal mask (which does not cover the individual's mouth and is generally considered to be less difficult to use during a sleep session) instead of a full-face mask. In a further example, the individual's weight loss may result in the individual only experiencing OSA when sleeping in certain positions (e.g., the individual now has positional OSA instead of OSA). In these examples, the updated treatment plan can include the use of a positional adjustment device that is configured to aid in causing the individual to sleep in a desired position during the sleep session. In general, an initial treatment plan can include a first group of settings for the respiratory therapy system, a first type of user interface, a first sleeping position, etc., and an updated treatment plan can include a second group of settings for the respiratory therapy system, a second type of user interface, a second sleeping position, etc.

In some implementations, the method 600 includes determining the body position of the individual when the first image data and/or the second image data was generated. The risk factor can be based at least in part on the body position. For example, the individual being in a certain body position may cause the values of certain physical features to appear to deviate from their actual values, which can impact the accuracy of the risk factor. Thus, by determining the body position of the individual, the risk factor can be adjusted as needed to account for the apparent deviations in the physical features. Any treatment plan for the individual can be based in part on the body position of the individual as well.

In some implementations, at least a portion of the first image data and/or the second image data can be generated while the individual is asleep during one or more sleep sessions. The image data can be analyzed to determine the body position of the individual during the sleep sessions, which in turn may affect any treatment plan recommended to the individual. For example, if the first image data and/or the second image data indicate that the individual is self-compensating during the sleep session (e.g., is subconsciously putting their body in a position to open their airway, such as with their head leaned back), the risk factor and/or the treatment plan can be updated. In this example, the treatment plan could include a recommendation to use a specific type of pillow during subsequent sleep sessions, a recommendation to sleep in a certain body position during subsequent sleep session, and/or other recommendations.

In some implementations, method 600 further comprises generating acoustic data that is representative of one or more sounds produced by the individual. The risk factor can be based at least in part on the acoustic data. The acoustic data can be analyzed to identify internal and/or external physical features, or to aid in identifying internal and/or external features in conjunction with the image data. In some implementations, analyzing the acoustic data includes determining the value of one or more acoustic features of the acoustic data (e.g., frequencies, amplitudes, spectrum, cepstrums, etc.), comparing the value of the acoustic features to baseline values, and identifying the physical features based on this comparison. In some implementations, the acoustic data is analyzed to determine a pronunciation or a change in pronunciation of one or more words and/or phrases by the individual. The pronunciation or change in pronunciation can be indicative of the risk factor and/or various physical features of the individual. In some implementations, the acoustic data can be analyzed to determine the tiredness level of the individual, which can be used to determine and/or adjust the risk factor. In some implementations, the acoustic data is generated via passive monitoring of the individual. In other cases, the acoustic data is generated after prompting the individual to produce one or more sounds (e.g., to say one or more desired words, phrases, sentences, etc.).

In some implementations, method 600 (and/or any of the various implementations of method 600 described herein) can be implemented using a system for determining a risk factor (such as system 10). The system includes a control system (such as control system 200 of system 10) and a memory (such as memory device 204 of system 10). The control system includes one or more processors (such as processor 202 of control system 200). The memory has stored thereon machine-readable instructions. The control system is coupled to the memory, and method 600 (and/or any of the various implementations of method 600 described herein) can be implemented when the machine-readable instructions in the memory are expected by at least one of the one or more processors of the control system.

Generally, method 600 can be implemented using a system (such as system 10) having a control system (such as control system 200 of system 10) with one or more processors (such as processor 202 of control system 200), and a memory (such as memory device 204 of system 10) storing machine-readable instructions. The control system can be coupled to the memory, and method 600 can be implemented when the machine-readable instructions are executed by at least one of the processors of the control system. Method 600 can also be implemented using a computer program product (such as a non-transitory computer readable medium) comprising instructions that when executed by a computer, cause the computer to carry out the steps of method 600.

### ALTERNATIVE IMPLEMENTATIONS

Alternative Implementation 1. A method for determining a risk factor for an individual that is associated with a condition, the method comprising: generating first image data of an interior of a mouth of the individual, an interior of a throat of the individual, or both, the first image data associated with one or more internal physical features of the individual; and determining the risk factor for the individual associated with the condition based at least in part on the first image data.

Alternative Implementation 2. The method of Alternative Implementation 1, wherein determining the risk factor includes determining that the individual currently has the condition.

Alternative Implementation 3. The method of Alternative Implementation 1 or Alternative Implementation 2, further comprising generating second image data of a head of the individual, a neck of the individual, or both, the second image data associated with one or more external physical features of the individual.

Alternative Implementation 4. The method of Alternative Implementation 3, wherein the risk factor is determined based on the first image data and the second image data.

Alternative Implementation 5. The method of Alternative Implementation 3, wherein the risk factor determined based at least in part on the first image data is an initial risk factor, and wherein the method further comprises updating the initial risk factor based at least in part on the second image data.

Alternative Implementation 6. The method of Alternative Implementation 5, wherein the updated risk factor is more accurate than the initial risk factor.

Alternative Implementation 7. The method of any one of Alternative Implementations 2 to 6, wherein the one or more external physical features of the individual are externally visible when the mouth of the individual is closed.

Alternative Implementation 8. The method of any one of Alternative Implementations 1 to 7, wherein the one or more internal physical features of the individual are not externally visible when the mouth of the individual is closed.

Alternative Implementation 9. The method of any one of Alternative Implementations 1 to 8, further comprising determining a contribution degree of (i) the one or more internal physical features, (ii) the one or more external physical features, or (iii) both (i) and (ii), the contribution degree of a respective physical feature being an estimate of an impact of the respective physical feature on a presence of the condition.

Alternative Implementation 10. The method of Alternative Implementation 9, wherein the estimate of the impact of the respective physical feature on the condition is an estimate of (i) a contribution of the respective physical feature on a development of the condition in the individual, (ii) a contribution of the respective physical feature on a severity of the condition in the individual, or (iii) both (i) and (ii).

Alternative Implementation 11. The method of Alternative Implementation 9 or Alternative Implementation 10, wherein the contribution degree of each respective physical feature is expressed (i) as a percentage, (ii) relative to the contribution degree of each other physical feature, or (iii) both (i) and (ii).

Alternative Implementation 12. The method of any one of Alternative Implementations 9 to 11, further comprising identifying at least one threshold physical feature having a contribution degree above a threshold value.

Alternative Implementation 13. The method of Alternative Implementation 12, further comprising determining whether each threshold physical feature is associated with (i) a tongue of the individual, a jaw of the individual, or both, or (ii) an airway of the individual.

Alternative Implementation 14. The method of any one of Alternative Implementations 9 to 13, further comprising identifying one or more modifiable physical features from the internal physical features and the external physical features, each of the one or more modifiable physical features being modifiable in response to (i) a change in a physical activity regimen of the individual, (ii) a change in a diet of the individual, (iii) a change in a medication regimen of the individual, or (iv) any combination of (i)-(iii).

Alternative Implementation 15. The method of Alternative Implementation 14, wherein each of the one or more modifiable physical features of the individual is an external physical feature or an internal physical feature.

Alternative Implementation 16. The method of Alternative Implementation 14 or Alternative Implementation 15, wherein the one or more modifiable physical features of the individual include a circumference of a neck of the individual, an amount of body fat in the head and neck area of the individual, a location of the body fat in the head and neck area of the individual, an amount of muscle in the head and neck area of the individual, a location of the muscle in the head and neck area of the individual, or any combination thereof.

Alternative Implementation 17. The method of any one of Alternative Implementations 9 to 16, further comprising identifying one or more non-modifiable physical features from the internal physical features and the external physical features that are not modifiable in response to (i) a change in a physical activity regimen of the individual, (ii) a change in a diet of the individual, (iii) a change in a medication regimen of the individual, or (iv) any combination of (i)-(iii).

Alternative Implementation 18. The method of Alternative Implementation 17, wherein the one or more non-modifiable physical features of the individual include a position of a jaw of the individual, a width of the jaw of the individual, a height of a tongue of the individual, a distance between a tongue of the individual and a roof of the mouth of the individual, a relative position between upper teeth of the individual and lower teeth of the individual, or any combination thereof.

Alternative Implementation 19. The method of any one of Alternative Implementations 12 to 18, further comprising determining a treatment plan for the individual based at least in part on the identified at least one physical feature having the contribution degree above the threshold value.

Alternative Implementation 20. The method of Alternative Implementation 19, wherein the treatment plan is further based on a severity of the condition.

Alternative Implementation 21. The method of Alternative Implementation 19 or Alternative Implementation 20, wherein: in response to the at least one threshold physical feature including one or more modifiable physical features, the determined treatment plan is a first treatment plan; and in response to the at least one threshold physical feature including no modifiable physical features, the determined treatment plan is a second treatment plan that is different than the first treatment plan.

Alternative Implementation 22. The method of any one of Alternative Implementations 1 to 21, further comprising determining a position of a body of the individual when the first image data is generated.

Alternative Implementation 23. The method of Alternative Implementation 22, wherein the risk factor is based at least in part on the position of the body of the individual.

Alternative Implementation 24. The method of Alternative Implementation 22 or Alternative Implementation 23, further comprising determining a treatment plan for the individual based at least in part on the position of the body of the individual.

Alternative Implementation 25. The method of Alternative Implementation 24, wherein the treatment plan for the individual is further based at least in part on the one or more internal physical features of the individual.

Alternative Implementation 26. The method of Alternative Implementation 24 or Alternative Implementation 25, wherein at least a portion of the first image data, the second image data, or both is generated during one or more sleep sessions of the individual, and wherein the treatment plan includes (i) a recommended type of pillow to use during one or more subsequent sleep sessions, (ii) a recommended body position to be in during the one or more subsequent sleep sessions, or (iii) both (i) and (ii).

Alternative Implementation 27. The method of any one of Alternative Implementations 1 to 26, wherein the first image data is generated at a first time, and wherein the method further comprises: generating additional first image data at a second time after the first time; and determining a change in the one or more external physical features of the individual based at least in part on the first image data and the additional first image data.

Alternative Implementation 28. The method of any one of Alternative Implementations 2 to 26, wherein the first image data and the second image data are generated at a first time, and wherein the method further comprises: generating additional first image data and second image data at a second time after the first time; and (i) determining a change in the one or more external physical features of the individual based at least in part on the first image data and the additional first image data, (ii) determining a change in the one or more internal physical features of the individual based at least in part on the second image data and the additional second image data, or (iii) both (i) and (ii).

Alternative Implementation 29. The method of Alternative Implementation 27 or Alternative Implementation 28, further comprising determining a change in the risk factor based at least in part on (i) the change in the one or more external physical features of the individual, (ii) the change in the one or more internal physical features of the individual, or (iii) both (i) and (ii).

Alternative Implementation 30. The method of any one of Alternative Implementations 27 to 29, further comprising: determining an initial treatment plan for the individual based at least in part on first image data, the second image data, or both; and determining an updated treatment plan based at least in part on (i) the change in the one or more external physical features of the individual, (ii) the change in the one or more internal physical features of the individual, or (iii) both (i) and (ii).

Alternative Implementation 31. The method of Alternative Implementation 30, wherein (i) the change in the one or more external physical features of the individual, (ii) the change in the one or more internal physical features of the individual, or (iii) both (i) and (ii) indicate that the individual experienced weight loss between the first time and the second time.

Alternative Implementation 32. The method of Alternative Implementation 31, wherein the initial treatment plan includes use of a respiratory therapy system with a first therapy pressure, and wherein the updated treatment plan includes use of the respiratory therapy system with a second therapy pressure that is less than the first therapy pressure.

Alternative Implementation 33. The method of Alternative Implementation 31 or Alternative Implementation 32, wherein the initial treatment plan includes use of a respiratory therapy system with a first type of user interface, and wherein the updated treatment plan includes use of the respiratory therapy system with a second type of user interface different than the first type of user interface.

Alternative Implementation 34. The method of Alternative Implementation 33, wherein the first type of user interface is a full-face mask, and the second type of user interface is a nasal mask.

Alternative Implementation 35. The method of any one of Alternative Implementations 31 to 34, wherein the initial treatment plan includes use of a respiratory therapy system, and wherein the updated treatment plan does not include use of the respiratory therapy system.

Alternative Implementation 36. The method of Alternative Implementation 35, wherein the updated treatment plan includes use of a positional adjustment device configured to aid in causing the individual to sleep in a desired position.

Alternative Implementation 37. The method of any one of Alternative Implementations 1 to 36, wherein determining the risk factor for the individual associated with the condition includes inputting the first image data, the second image data, or both into a trained machine learning model, the machine learning model being configured to output the risk factor.

Alternative Implementation 38. The method of any one of Alternative Implementations 1 to 37, further comprising generating acoustic data representative of one or more sounds produced by the individual, wherein the risk factor is based at least in part on the acoustic data.

Alternative Implementation 39. The method of Alternative Implementation 38, further comprising analyzing the acoustic data to identify the one or more external physical features of the individual, the one or more internal physical features of the individual, or both.

Alternative Implementation 40. The method of Alternative Implementation 38 or Alternative Implementation 39, further comprising: analyzing the acoustic data to determine a value of one or more acoustic features of the acoustic data; comparing the value of each of the one or more acoustic features to a baseline value; and based at least in part on the comparison, identifying the one or more external physical features of the individual, the one or more internal physical features of the individual, or both.

Alternative Implementation 41. The method of any one of Alternative Implementations 38 to 40, further comprising analyzing the acoustic data to determine a pronunciation by the individual of at least one of the one or more sounds, the pronunciation being indicative of the risk factor.

Alternative Implementation 42. The method of any one of Alternative Implementations 38 to 41, further comprising analyzing the acoustic data to determine a change in a pronunciation by the individual of at least one of the one or more sounds, the change in the pronunciation being indicative of the risk factor.

Alternative Implementation 43. The method of any one of Alternative Implementations 38 to 42, further comprising analyzing the acoustic data to determine a tiredness level of the individual, the tiredness level of the individual being indicative of the risk factor.

Alternative Implementation 44. The method of any one of Alternative Implementations 38 to 43, wherein at least a portion of the acoustic data is generated via passive monitoring of the individual.

Alternative Implementation 45. The method of any one of Alternative Implementations 38 to 44, wherein at least a portion of the acoustic data is generated after prompting the individual to produce at least one of the one or more sounds.

Alternative Implementation 46. The method of any one of Alternative Implementations 38 to 45, wherein the one or more sounds includes one or more words, one or more phrases, one or more sentences, or any combination thereof.

Alternative Implementation 47. A system for determining a risk factor for a condition, the system comprising: a control system including one or more processors; and a memory having stored thereon machine-readable instructions; wherein the control system is coupled to the memory, and the method of any one of Alternative Implementations 1 to 46 is implemented when the machine-readable instructions in the memory are executed by at least one of the one or more processors of the control system.

Alternative Implementation 48. A system for determining a risk factor for a condition, the system including a control system having one or more processors configured to implement the method of any one of Alternative Implementations 1 to 46.

Alternative Implementation 49. A computer program product comprising instructions which, when executed by a computer, cause the computer to carry out the method of any one of Alternative Implementations 1 to 46.

Alternative Implementation 50. The computer program product of Alternative Implementation 49, wherein the computer program product is a non-transitory computer readable medium.

Alternative Implementation 51. A system for determining a risk factor for an individual that is associated with a condition, the system comprising:
an electronic interface configured to generate data associated with the individual, receive data associated with the individual, or both;
a memory storing machine-readable instructions; and
a control system including one or more processors configured to execute the machine-readable instructions to:
generate first image data of an interior of a mouth of an individual, an interior of a throat of the individual, or both, the first image data associated with one or more internal physical features of the individual; and
determine the risk factor for the individual associated with the condition based at least in part on the first image data.

Alternative Implementation 52. The system of Alternative Implementation 51, wherein determining the risk factor includes determining that the individual currently has the condition.

Alternative Implementation 53. The system of Alternative Implementation 51 or Alternative Implementation 52, wherein the one or more processors are further configured to execute the machine-readable instructions to generate second image data of a head of the individual, a neck of the individual, or both, the second image data associated with one or more external physical features of the individual.

Alternative Implementation 54. The system of Alternative Implementation 53, wherein the risk factor is determined based on the first image data and the second image data.

Alternative Implementation 55. The system of Alternative Implementation 53, wherein the risk factor determined based at least in part on the first image data is an initial risk factor, and wherein the one or more processors are further configured to execute the machine-readable instructions to update the initial risk factor based at least in part on the second image data.

Alternative Implementation 56. The system of Alternative Implementation 55, wherein the updated risk factor is more accurate than the initial risk factor.

Alternative Implementation 57. The system of any one of Alternative Implementations 52 to 56, wherein the one or more external physical features of the individual are externally visible when the mouth of the individual is closed.

Alternative Implementation 58. The system of any one of Alternative Implementations 51 to 57, wherein the one or more internal physical features of the individual are not externally visible when the mouth of the individual is closed.

Alternative Implementation 59. The system of any one of Alternative Implementations 51 to 58, wherein the one or more processors are further configured to execute the machine-readable instructions to determine a contribution degree of (i) the one or more internal physical features, (ii) the one or more external physical features, or (iii) both (i) and (ii), the contribution degree of a respective physical feature being an estimate of an impact of the respective physical feature on a presence of the condition.

Alternative Implementation 60. The system of Alternative Implementation 59, wherein the estimate of the impact of the respective physical feature on the condition is an estimate of (i) a contribution of the respective physical feature on a development of the condition in the individual, (ii) a contribution of the respective physical feature on a severity of the condition in the individual, or (iii) both (i) and (ii).

Alternative Implementation 61. The system of Alternative Implementation 59 or Alternative Implementation 60, wherein the contribution degree of each respective physical feature is expressed (i) as a percentage, (ii) relative to the contribution degree of each other physical feature, or (iii) both (i) and (ii).

Alternative Implementation 62. The system of any one of Alternative Implementations 59 to 61, wherein the one or more processors are further configured to execute the machine-readable instructions to identify at least one threshold physical feature having a contribution degree above a threshold value.

Alternative Implementation 63. The system of Alternative Implementation 62, wherein the one or more processors are further configured to execute the machine-readable instructions to determine whether each threshold physical feature is associated with (i) a tongue of the individual, a jaw of the individual, or both, or (ii) an airway of the individual.

Alternative Implementation 64. The system of any one of Alternative Implementations 59 to 63, wherein the one or more processors are further configured to execute the machine-readable instructions to identify one or more modifiable physical features from the internal physical features and the external physical features, each of the one or more modifiable physical features being modifiable in response to (i) a change in a physical activity regimen of the individual, (ii) a change in a diet of the individual, (iii) a change in a medication regimen of the individual, or (iv) any combination of (i)-(iii).

Alternative Implementation 65. The system of Alternative Implementation 64, wherein each of the one or more modifiable physical features of the individual is an external physical feature or an internal physical feature.

Alternative Implementation 66. The system of Alternative Implementation 64 or Alternative Implementation 65, wherein the one or more modifiable physical features of the individual include a circumference of a neck of the individual, an amount of body fat in the head and neck area of the individual, a location of the body fat in the head and neck area of the individual, an amount of muscle in the head and neck area of the individual, a location of the muscle in the head and neck area of the individual, or any combination thereof.

Alternative Implementation 67. The system of any one of Alternative Implementations 59 to 66, wherein the one or more processors are further configured to execute the machine-readable instructions to identify one or more non-modifiable physical features from the internal physical features and the external physical features that are not modifiable in response to (i) a change in a physical activity regimen of the individual, (ii) a change in a diet of the individual, (iii) a change in a medication regimen of the individual, or (iv) any combination of (i)-(iii).

Alternative Implementation 68. The system of Alternative Implementation 67, wherein the one or more non-modifiable physical features of the individual include a position of a jaw of the individual, a width of the jaw of the individual, a height of a tongue of the individual, a distance between a tongue of the individual and a roof of the mouth of the individual, a relative position between upper teeth of the individual and lower teeth of the individual, or any combination thereof.

Alternative Implementation 69. The system of any one of Alternative Implementations 62 to 68, wherein the one or more processors are further configured to execute the machine-readable instructions to determine a treatment plan for the individual based at least in part on the identified at least one physical feature having the contribution degree above the threshold value.

Alternative Implementation 70. The system of Alternative Implementation 69, wherein the treatment plan is further based on a severity of the condition.

Alternative Implementation 71. The system of Alternative Implementation 69 or Alternative Implementation 70, wherein: in response to the at least one threshold physical feature including one or more modifiable physical features, the determined treatment plan is a first treatment plan; and in response to the at least one threshold physical feature including no modifiable physical features, the determined treatment plan is a second treatment plan that is different than the first treatment plan.

Alternative Implementation 72. The system of any one of Alternative Implementations 51 to 71, wherein the one or more processors are further configured to execute the machine-readable instructions to determine a position of a body of the individual when the first image data is generated.

Alternative Implementation 73. The system of Alternative Implementation 72, wherein the risk factor is based at least in part on the position of the body of the individual.

Alternative Implementation 74. The system of Alternative Implementation 72 or Alternative Implementation 73, wherein the one or more processors are further configured to execute the machine-readable instructions to determine a treatment plan for the individual based at least in part on the position of the body of the individual.

Alternative Implementation 75. The system of Alternative Implementation 74, wherein the treatment plan for the individual is further based at least in part on the one or more internal physical features of the individual.

Alternative Implementation 76. The system of Alternative Implementation 74 or Alternative Implementation 75, wherein at least a portion of the first image data, the second image data, or both is generated during one or more sleep sessions of the individual, and wherein the treatment plan includes (i) a recommended type of pillow to use during one or more subsequent sleep sessions, (ii) a recommended body position to be in during the one or more subsequent sleep sessions, or (iii) both (i) and (ii).

Alternative Implementation 77. The system of any one of Alternative Implementations 51 to 76, wherein the first image data is generated at a first time, and wherein the one or more processors are further configured to execute the machine-readable instructions to: generate additional first image data at a second time after the first time; and determine a change in the one or more external physical features of the individual based at least in part on the first image data and the additional first image data.

Alternative Implementation 78. The system of any one of Alternative Implementations 52 to 76, wherein the first image data and the second image data are generated at a first time, and wherein the one or more processors are further configured to execute the machine-readable instructions to: generate additional first image data and second image data at a second time after the first time; and (i) determine a change in the one or more external physical features of the individual based at least in part on the first image data and the additional first image data, (ii) determining a change in the one or more internal physical features of the individual based at least in part on the second image data and the additional second image data, or (iii) both (i) and (ii).

Alternative Implementation 79. The system of Alternative Implementation 77 or Alternative Implementation 78, wherein the one or more processors are further configured to execute the machine-readable instructions to determine a change in the risk factor based at least in part on (i) the change in the one or more external physical features of the individual, (ii) the change in the one or more internal physical features of the individual, or (iii) both (i) and (ii).

Alternative Implementation 80. The system of any one of Alternative Implementations 77 to 79, wherein the one or more processors are further configured to execute the machine-readable instructions to: determine an initial treatment plan for the individual based at least in part on first image data, the second image data, or both; and determine an updated treatment plan based at least in part on (i) the change in the one or more external physical features of the individual, (ii) the change in the one or more internal physical features of the individual, or (iii) both (i) and (ii).

Alternative Implementation 81. The system of Alternative Implementation 80, wherein (i) the change in the one or more external physical features of the individual, (ii) the change in the one or more internal physical features of the individual, or (iii) both (i) and (ii) indicate that the individual experienced weight loss between the first time and the second time.

Alternative Implementation 82. The system of Alternative Implementation 81, wherein the initial treatment plan includes use of a respiratory therapy system with a first therapy pressure, and wherein the updated treatment plan includes use of the respiratory therapy system with a second therapy pressure that is less than the first therapy pressure.

Alternative Implementation 83. The system of Alternative Implementation 81 or Alternative Implementation 82, wherein the initial treatment plan includes use of a respiratory therapy system with a first type of user interface, and wherein the updated treatment plan includes use of the respiratory therapy system with a second type of user interface different than the first type of user interface.

Alternative Implementation 84. The system of Alternative Implementation 83, wherein the first type of user interface is a full-face mask, and the second type of user interface is a nasal mask.

Alternative Implementation 85. The system of any one of Alternative Implementations 81 to 84, wherein the initial treatment plan includes use of a respiratory therapy system, and wherein the updated treatment plan does not include use of the respiratory therapy system.

Alternative Implementation 86. The system of Alternative Implementation 85, wherein the updated treatment plan includes use of a positional adjustment device configured to aid in causing the individual to sleep in a desired position.

Alternative Implementation 87. The system of any one of Alternative Implementations 51 to 86, wherein determining the risk factor for the individual associated with the condition includes inputting the first image data, the second image data, or both into a trained machine learning model, the machine learning model being configured to output the risk factor.

Alternative Implementation 88. The system of any one of Alternative Implementations 51 to 87, wherein the one or more processors are further configured to execute the machine-readable instructions to generate acoustic data representative of one or more sounds produced by the individual, wherein the risk factor is based at least in part on the acoustic data.

Alternative Implementation 89. The system of Alternative Implementation 88, wherein the one or more processors are further configured to execute the machine-readable instructions to analyze the acoustic data to identify the one or more external physical features of the individual, the one or more internal physical features of the individual, or both.

Alternative Implementation 90. The system of Alternative Implementation 88 or Alternative Implementation 89, wherein the one or more processors are further configured to execute the machine-readable instructions to: analyze the acoustic data to determine a value of one or more acoustic features of the acoustic data; compare the value of each of the one or more acoustic features to a baseline value; and based at least in part on the comparison, identify the one or more external physical features of the individual, the one or more internal physical features of the individual, or both.

Alternative Implementation 91. The system of any one of Alternative Implementations 88 to 90, wherein the one or more processors are further configured to execute the machine-readable instructions to analyze the acoustic data to determine a pronunciation by the individual of at least one of the one or more sounds, the pronunciation being indicative of the risk factor.

Alternative Implementation 92. The system of any one of Alternative Implementations 88 to 91, wherein the one or more processors are further configured to execute the machine-readable instructions to analyze the acoustic data to determine a change in a pronunciation by the individual of at least one of the one or more sounds, the change in the pronunciation being indicative of the risk factor.

Alternative Implementation 93. The system of any one of Alternative Implementations 88 to 92, wherein the one or more processors are further configured to execute the machine-readable instructions to analyze the acoustic data to determine a tiredness level of the individual, the tiredness level of the individual being indicative of the risk factor.

Alternative Implementation 94. The system of any one of Alternative Implementations 88 to 93, wherein at least a portion of the acoustic data is generated via passive monitoring of the individual.

Alternative Implementation 95. The system of any one of Alternative Implementations 88 to 94, wherein at least a portion of the acoustic data is generated after prompting the individual to produce at least one of the one or more sounds.

Alternative Implementation 96. The system of any one of Alternative Implementations 88 to 95, wherein the one or more sounds includes one or more words, one or more phrases, one or more sentences, or any combination thereof.

One or more elements or aspects or steps, or any portion(s) thereof, from one or more of any of the Alternative Implementations and/or claims herein can be combined with one or more elements or aspects or steps, or any portion(s) thereof, from one or more of any of the other Alternative Implementations and/or claims herein or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1-50. (canceled)

51. A system for determining a risk factor for an individual that is associated with a condition, the system comprising:
an electronic interface configured to generate data associated with the individual, receive data associated with the individual, or both;
a memory storing machine-readable instructions; and
a control system including one or more processors configured to execute the machine-readable instructions to:
generate first image data of an interior of a mouth of an individual, an interior of a throat of the individual, or both, the first image data associated with one or more internal physical features of the individual; and
determine the risk factor for the individual associated with the condition based at least in part on the first image data.

52. The system of claim 51, wherein determining the risk factor includes determining that the individual currently has the condition.

53. The system of claim 51, wherein the one or more processors are further configured to execute the machine-readable instructions to generate second image data of a head of the individual, a neck of the individual, or both, the second image data associated with one or more external physical features of the individual, and wherein the risk factor is determined based on the first image data and the second image data.

54. (canceled)

55. The system of claim 53, wherein the risk factor determined based at least in part on the first image data is an initial risk factor, and wherein the one or more processors are further configured to execute the machine-readable instructions to update the initial risk factor based at least in part on the second image data.

56-58. (canceled)

59. The system of claim 53, wherein the one or more processors are further configured to execute the machine-readable instructions to determine a contribution degree of (i) the one or more internal physical features, (ii) the one or more external physical features, or (iii) both (i) and (ii), the contribution degree of a respective physical feature being an estimate of an impact of the respective physical feature on a presence of the condition.

60-61. (canceled)

62. The system of claim 59, wherein the one or more processors are further configured to execute the machine-readable instructions to:
identify at least one threshold physical feature having a contribution degree above a threshold value; and
determine whether each threshold physical feature is associated with (i) a tongue of the individual, a jaw of the individual, or both, or (ii) an airway of the individual.

63. (canceled)

64. The system of claim 59, wherein the one or more processors are further configured to execute the machine-readable instructions to identify one or more modifiable physical features from the internal physical features and the external physical features, each of the one or more modifiable physical features being modifiable in response to (i) a change in a physical activity regimen of the individual, (ii) a change in a diet of the individual, (iii) a change in a medication regimen of the individual, or (iv) any combination of (i)-(iii).

65. (canceled)

66. The system of claim 64, wherein the one or more modifiable physical features of the individual include a circumference of a neck of the individual, an amount of body fat in the head and neck area of the individual, a location of the body fat in the head and neck area of the individual, an amount of muscle in the head and neck area of the individual, a location of the muscle in the head and neck area of the individual, or any combination thereof.

67. The system of claim 59, wherein the one or more processors are further configured to execute the machine-readable instructions to identify one or more non-modifiable physical features from the internal physical features and the external physical features that are not modifiable in response to (i) a change in a physical activity regimen of the individual, (ii) a change in a diet of the individual, (iii) a change in a medication regimen of the individual, or (iv) any combination of (i)-(iii).

68. The system of claim 67, wherein the one or more non-modifiable physical features of the individual include a position of a jaw of the individual, a width of the jaw of the individual, a height of a tongue of the individual, a distance between a tongue of the individual and a roof of the mouth of the individual, a relative position between upper teeth of the individual and lower teeth of the individual, or any combination thereof.

69. The system of claim 62, wherein the one or more processors are further configured to execute the machine-readable instructions to determine a treatment plan for the individual based at least in part on the identified at least one physical feature having the contribution degree above the threshold value.

70. (canceled)

71. The system of claim 69, wherein:
in response to the at least one threshold physical feature including one or more modifiable physical features, the determined treatment plan is a first treatment plan; and
in response to the at least one threshold physical feature including no modifiable physical features, the determined treatment plan is a second treatment plan that is different than the first treatment plan.

72. The system of claim 51, wherein the one or more processors are further configured to execute the machine-readable instructions to determine a position of a body of the individual when the first image data is generated, and wherein the risk factor is based at least in part on the position of the body of the individual.

73-75. (canceled)

76. The system of claim 72, wherein at least a portion of the first image data is generated during one or more sleep sessions of the individual, and wherein the treatment plan includes (i) a recommended type of pillow to use during one or more subsequent sleep sessions, (ii) a recommended body position to be in during the one or more subsequent sleep sessions, or (iii) both (i) and (ii).

77. The system of claim 53, wherein the first image data is generated at a first time, and wherein the one or more processors are further configured to execute the machine-readable instructions to:
generate additional first image data at a second time after the first time;
determine a change in the one or more external physical features of the individual based at least in part on the first image data and the additional first image data; and
determine a change in the risk factor based at least in part on the change in the one or more external physical features of the individual.

78. The system of claim 77, wherein the first image data and the second image data are generated at a first time, and wherein the one or more processors are further configured to execute the machine-readable instructions to:
generate additional first image data and second image data at a second time after the first time;
(i) determine a change in the one or more external physical features of the individual based at least in part on the first image data and the additional first image data, (ii) determining a change in the one or more internal physical features of the individual based at least in part on the second image data and the additional second image data, or (iii) both (i) and (ii); and
determine a change in the risk factor based at least in part on (i) the change in the one or more external physical features of the individual, (ii) the change in the one or more internal physical features of the individual, or (iii) both (i) and (ii).

79. (canceled)

80. The system of claim 78, wherein the one or more processors are further configured to execute the machine-readable instructions to:
determine an initial treatment plan for the individual based at least in part on first image data, the second image data, or both; and
determine an updated treatment plan based at least in part on (i) the change in the one or more external physical features of the individual, (ii) the change in the one or more internal physical features of the individual, or (iii) both (i) and (ii),
wherein the initial treatment plan includes use of a respiratory therapy system with a first therapy pressure, use of the respiratory therapy system with a first type of user interface, or both, and wherein the updated treatment plan includes use of the respiratory therapy system with a second therapy pressure that is less than the first therapy pressure, use of the respiratory therapy system with a second type of user interface different than the first type of user interface, use of a positional adjustment device configured to aid in causing the individual to sleep in a desired position, or any combination thereof.

81-86. (canceled)

87. The system of claim 53, wherein determining the risk factor for the individual associated with the condition includes inputting the first image data, the second image data, or both into a trained machine learning model, the machine learning model being configured to output the risk factor.

88. The system of claim 53, wherein the one or more processors are further configured to execute the machine-readable instructions to generate acoustic data representative of one or more sounds produced by the individual and to analyze the acoustic data to identify the one or more external physical features of the individual, the one or more internal physical features of the individual, or both, wherein the risk factor is based at least in part on the acoustic data.

89-90. (canceled)

91. The system of claim 88, wherein:
the one or more processors are further configured to execute the machine-readable instructions to analyze the acoustic data to determine a pronunciation by the individual of at least one of the one or more sounds, the pronunciation being indicative of the risk factor; or
the one or more processors are further configured to execute the machine-readable instructions to analyze the acoustic data to determine a change in a pronunciation by the individual of at least one of the one or more sounds, the change in the pronunciation being indicative of the risk factor; or
the one or more processors are further configured to execute the machine-readable instructions to analyze the acoustic data to determine a tiredness level of the individual, the tiredness level of the individual being indicative of the risk factor.

92-96. (canceled)
